(19) United States
(12) Patent Application Publication
(10) Pub. No.: US 2008/0154100 A1
(43) Pub. Date: Jun. 26, 2008

(54) Patient monitoring apparatus for determining a parameter representing an intrathoracic volume compartment of a patient

(75) Inventors: Thomas Thalmeier, Dorfen (DE); Oliver Godje, Deining (DE); Jorg Scheier, Munich (DE)

Correspondence Address: COLLARD & ROE, P.C., 1077 NORTHERN BOULEVARD, ROSLYN, NY, 11576, US

(73) Assignee: PULSION Medical Systems AG

(21) Appl. No.: 12/004,298

(22) Filed: Dec. 20, 2007

Related U.S. Application Data
(60) Provisional application No. 60/876,976, filed on Dec. 22, 2006.

(30) Foreign Application Priority Data
Dec. 22, 2006 (EP) 06 127 135.9

(51) Int. Cl. A61B 5/00

(52) U.S. Cl. 600/301

(57) ABSTRACT

A patient monitoring apparatus determines a parameter representing an intrathoracic volume compartment, such as extravascular lung water, and normalizes the determined parameter on the basis of input biometric data, such as body height, using an algorithm selected from a plurality of algorithms depending on a category a patient is allocated to. Preferably, the parameter is normalized by dividing it by a predicted (ideal) body weight which is calculated from the patient's body height. A memory of the patient monitoring apparatus has stored therein a program for selectively executing one of several algorithms for determining a predicted weight. Before determining normalized values, the operator is prompted to input height and gender of the patient and whether the patient is an adult or a pediatric patient.

### CROSS REFERENCE TO RELATED APPLICATIONS

Applicants claim priority under 35 U.S.C. §119 of European Patent Application No. 06 127 135.9 filed Dec. 22, 2006. Applicants also claim priority under 35 U.S.C. §119(e)(1) and the benefit of U.S. Provisional Application Ser. No. 60/876,976, filed on Dec. 22, 2006 which is incorporated by reference herein.

The present invention relates to patient monitoring apparatus, in particular to patient monitoring apparatus for determining a parameter representing an intrathoracic volume compartment of a monitored patient.

Patient monitoring apparatus are commonly used in modern day hospitals for monitoring the condition of the circulatory system of critically ill patients. As is well known to the person skilled in the art, patient monitoring apparatus may function according to one of a variety of measurement and evaluation principles, such as (right heart or transpulmonary) thermodilution, dye dilution, pulse contour analysis, or may combine two or more of these measurement and evaluation principles. Applying suitable methods according to these principles yields a variety of parameters which enable the physician in charge to judge the present condition of the patient and to take appropriate counter measures, if the condition should worsen. Particularly important among these parameters are intrathoracic volume compartments (often also referred to simply as intrathoracic volumes) such as global enddiastolic volume GEDV, extravascular lung water EVLW, pulmonary blood volume PBV and intrathoracic blood volume ITBV. For example, GEDV is used to assess the filling state of a patient, and EVLW is an important parameter for observing the development of a pulmonary oedema.

Patient monitoring apparatus for determining one or more of the above parameters are described inter alii in U.S. Pat. No. 5,526,817, U.S. Pat. No. 6,394,961, U.S. Pat. No. 6,537,230 and U.S. Pat. No. 6,736,782.

Before drawing medical conclusions from the value of a specific intrathoracic volume compartment it is often necessary to take into consideration the body dimensions of a particular patient. For example, the identical value of extravascular lung water may indicate the development of an edema when determined for a small patient, whereas it may suggest a noncritical situation when determined for a large patient. Therefore, extravascular lung water is often normalised (or indexed) by dividing it by the patient's actual weight. However, it has been found that considerable deviations may occur in the case of very corpulent or very lean patients. Such deviations can cause errors in a diagnosis based on the normalised (or indexed) value.

In view of the above, it is an object of the present invention to technically improve the provision of intrathoracic volume data determined by a patient monitoring apparatus as a starting point for further medical diagnosis in order to provide a basis for facilitated (and thus more reliable) diagnostic interpretation.

According to one aspect of the present invention, this object is accomplished by providing a patient monitoring apparatus according to claim 1. Advantageous embodiments of the present invention can be configured according to any of claims 2-14.

According to another aspect of the present invention, this object is accomplished by providing a method according to claim 15. Advantageous embodiments of this method can be carried out according to any of claims 16-25.

According to the present invention, a parameter representing an intrathoracic volume compartment is thus normalised on the basis of input biometric data, such as body height, using an algorithm selected from a plurality of algorithms depending on a category a patient is allocated to. The categorizing information, on which this allocation is based upon, may be taken from a set of discrete data determining a category directly (such as gender, adult patient/pediatric patient, underweight/overweight/neither underweight nor overweight) or from a continuous data range such as patient's age, waist circumference or body height. Input biometric information may thus also be used as categorizing information.

For ease of reading only, parameters representing an intrathoracic volume compartment of a patient will also be referred to as intrathoracic volume parameters hereinafter.

The selection of an algorithm, according to the present invention, can be implemented in many different ways, such as
selecting, from a plurality of prestored program modules or sub routines, a program module or sub routine, respectively, to be carried out, or
substituting coefficients and/or constants in a calculation formula or correction function,
   or the like.

In fact, any branch in a course of calculation implemented for normalising an intrathoracic volume parameter, wherein input categorizing data determines which calculation path is to be followed, is to be considered a selection of algorithms according to the present invention.

The present invention makes it thus possible to reduce or even eliminate errors induced by considering intrathoracic volume parameters without taking into account a patient's actual build. In fact, by applying a patient category dependent normalisation algorithm, the present invention automatically includes the consideration of general differences that are to be expected between two individuals belonging to a different category (e.g. a different age group or gender), even if they share the same biometric datum (such as body height), on which normalisation is based.

According to a particularly preferred embodiment of the present invention, one or more of the algorithms implemented for normalising an intrathoracic volume parameter is an algorithm (or are algorithms, respectively) for determining a notional (or virtual) property of the patient from the input biometric data. Such notional (or virtual) properties may include, for example, a notional weight (such as determined by known formulas for height dependent and/or age dependent and/or gender dependent “predicted weight” or “ideal weight”) or a notional body surface area. The latter may be determined, for example, by a known formula for body surface area, such as published in Haycock, G. B., Schwartz, G. J., Wisotsky, D. H.: “Geometric method for measuring body surface area: a height-weight formula validated in infants, children, and adults.” The Journal of Pediatrics 93 (1978), 62-66 and in Du Bois, D., Du Bois, E.: “A formula to estimate the approximate surface area if height and weight be known.” The Archives of Internal Medicine 121 (1916), 863-872, wherein, however, patient's weight is substituted by patient's notional weight.

If, for example, extra vascular lung water of an overweight patient is normalised using his “predicted weight” or “ideal weight” instead of his actual body weight, values will be correctly perceived to be dangerous with regard to development of an edema, which otherwise may have been erroneously perceived to be normal.

Generally, any of the embodiments described or options mentioned herein may be particularly advantageous depending on the actual conditions of application. Further, features of one embodiment may be combined with features of another embodiment as well as features known per se from the prior art as far as technically possible and unless indicated otherwise. It goes without saying that the present invention may be advantageously implemented for determining not just one but a plurality of parameters respectively representing various intrathoracic volume compartments of a monitored patient some or all of which can be normalized in the inventive manner.

The accompanying drawings, which are schematic illustrations, serve for a better understanding of the features of the present invention.

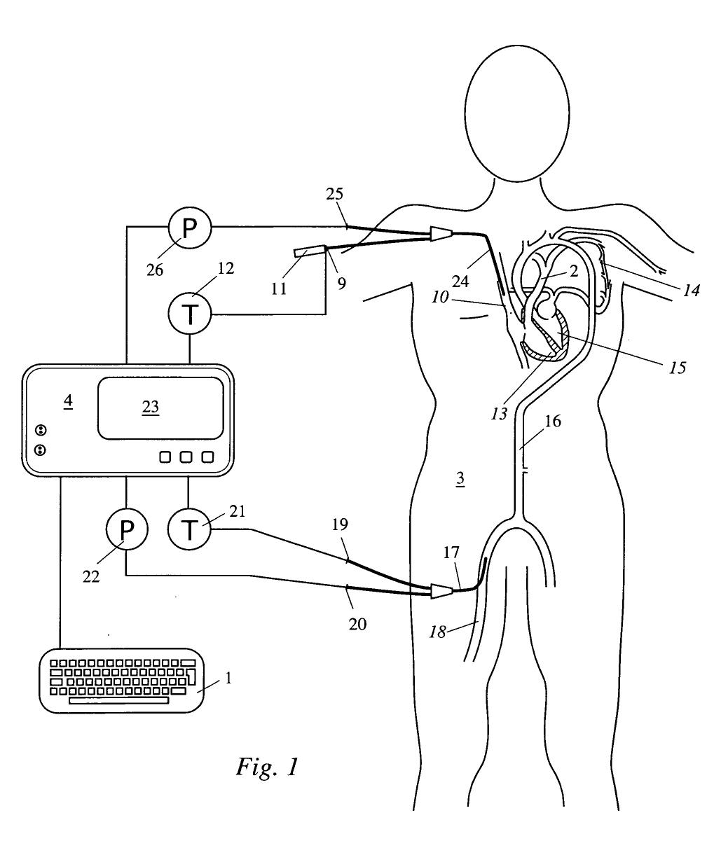

FIG. 1 schematically illustrates an example of a patient monitoring apparatus according to the present invention wherein determination of parameters representing intrathoracic volume compartments is based on transpulmonary thermodilution measurements.

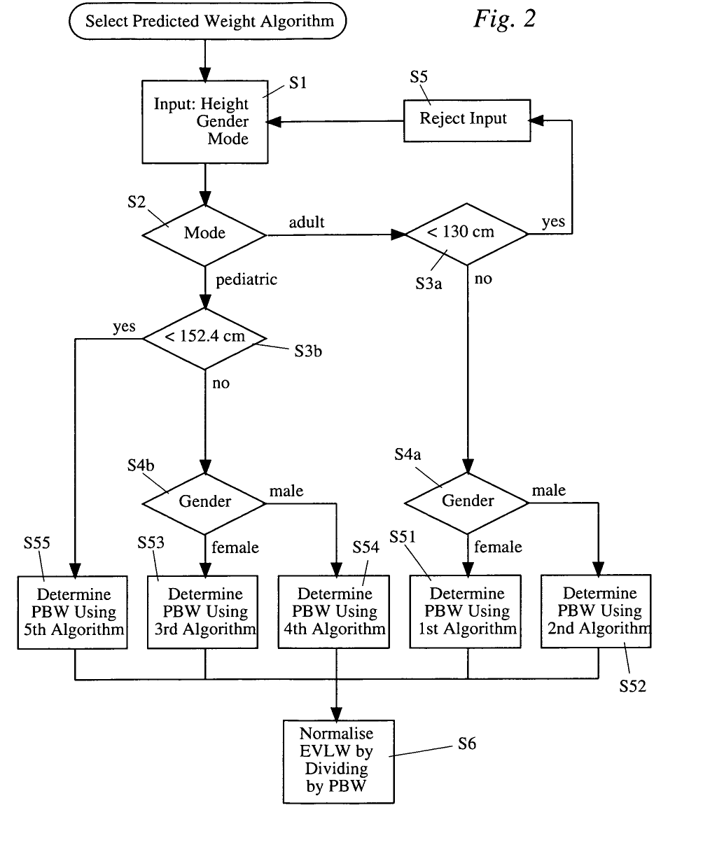

FIG. 2 illustrates an examplary procedure of selecting a predicted weight determining algorithm in accordance with the present invention.

In addition to hardware and software for transpulmonary thermodilution measurements, the patient monitoring apparatus depicted in FIG. 1 is further equipped with hardware and software for performing pulse contour analysis. Generally, the present invention is not limited to such a setup, but may be implemented using hardware and software for determining parameters representing intrathoracic volume compartments on the basis of dye dilution, combined thermodilution and dye dilution, or other known techniques.

The apparatus comprises a central venous catheter 24 extending into a central vein 10 of the patient 3. The central venous catheter 24 comprises a proximal port 9 connected with a lumen, the distal opening of which is located in the central vein (vena cava superior) 10. This lumen is used for injecting a bolus with a temperature different (usually lower) from the patient's blood temperature, thus introducing a travelling temperature deviation to the blood stream. As an alternative, a temperature deviation may be introduced by local heating or cooling in the central vein using a catheter equipped with heating (such as a heating coil) or cooling means (such as a Peltier-element), respectively. A thermodilution setup and a central venous catheter assembly equipped with means for local heating of central venous blood, which can be implemented correspondingly in a right heart catheter 1 assembly as well, are described in U.S. Pat. No. 6,736,782.

Preferably, the proximal port 9 is equipped with an injection channel 11 for accomplishing a bolus injection as well-defined as possible in terms of time and duration of bolus injection as well as bolus temperature. For this, the injection channel 11 may comprise a pressure switch and a temperature sensor connected with the patient monitor 4 via a transducer 12. The injection channel may be configured as described in U.S. Pat. No. 6,491,640.

Optionally, the central venous catheter 24 may be equipped with a distal pressure sensor for measuring blood pressure in the central vein 10. The sensor signal is transmitted to the patient monitor 4 via a proximal catheter port 5 and transducer 26.

The system further comprises an arterial catheter 17 comprising a temperature sensor for measuring the local blood temperature in an artery 18 and a pressure sensor for measuring the arterial blood pressure. Each of the sensors is connected with the patient monitor 4 via proximal catheter ports 19, 20 and transducers 21, 22, respectively. Though the arterial catheter 17 is placed in a femoral artery in the schematic view of FIG. 1, catheter placement in other arteries such as axillary (brachial) artery or radial artery may also be suitable.

The temperature deviation introduced to the patient's 3 blood stream by bolus injection or local heating dilutes while travelling through the right heart 13, pulmonary circulation 14, left heart 15 and systemic circulation 16. This dilution is evaluated by applying known thermodilution algorithms using temperature over time measurements performed with the temperature sensor of the arterial catheter 17. The patient monitor 4 is adapted to perform this evaluation using an evaluation program stored in a memory of the patient monitor 4.

In particular, global enddiastolic volume GEDV is determined in the following manner.

GEDV=ITTV−PTV

wherein ITTV is the intrathoracic thermo volume and PTV is the pulmonary thermo volume. These parameters are determined as follows

ITTV=CO·MTt_{TDa}

PTV=CO·DSt_{TDa}

wherein MTt_{TDa} is the mean transit time and DSt_{TDa} is the downslope time (i.e. the time the blood temperature difference ΔT_{B}(t) takes to drop by the factor 1/e where the dilution curve shows exponential decay) both determined from the dilution curve measured by the transpulmonary setup. CO is the cardiac output and may be determined either by pulse contour analysis using known algorithms (such as disclosed in U.S. Pat. No. 6,315,735) based on the pressure-over-time signal measured with the pressure sensor of the arterial catheter 17, or it may be determined (using the temperature-over-time signal measured with the temperature sensor of the arterial catheter 17) by known thermodilution algorithms based on the Stewart-Hamilton equation

$$CO = \frac{{V}_{L} ( {T}_{B} - {T}_{L} ) {K}_{1} {K}_{2}}{\int Δ {T}_{B} ( t ) d t}$$

wherein T_{B} is the initial blood temperature, T_{L} is the temperature of the liquid bolus, which is used as thermal indicator, V_{L} is the thermal indicator volume, K1 and K2 are constants to consider the specific measurement setup, and ΔT_{B}(t) is the blood temperature as a function of time with respect to the baseline blood temperature T_{B}.

A parameter representing extravascular lung water EVLW can be determined as the difference between Intrathoracic Thermovolume ITTV and the Intrathoracic blood volume ITBV

EVLW=ITTV−ITBV

In the present embodiment, EVLW is normalised with a predicted (ideal) body weight PBW which is calculated from the patient's 3 body height:

EVLWi=EVLW/PBW

wherein EVLWi is the normalised value.

The memory of the patient monitor 4 has stored therein a program for selectively executing one of six algorithms for determining a predicted weight.
The first algorithm is optimized for determining the predicted weight of a female adult with a height above a first threshold value of patient's height.
The second algorithm is optimized for determining the predicted weight of a male adult with a height above a second threshold value of patient's height.
The third algorithm is optimized for determining the predicted weight of a female pediatric patient with a height above a third threshold value of patient's height.
The fourth algorithm is optimized for determining the predicted weight of a male pediatric patient with a height above a fourth threshold value of patient's height.
The fifth algorithm is optimized for determining the predicted weight of a female pediatric patient with a height below the third threshold value of patient's height.
The sixth algorithm is optimized for determining the predicted weight of a male pediatric patient with a height below the fourth threshold value of patient's height.

As an example, predicted weight algorithms may be based on available validated formulas derived from well founded empiric data from The Acute Respiratory Distress Syndrome Network: “Ventilation With Lower Tidal Volumes As Compared With Traditional Tidal Volumes For Acute Lung Injury And The Acute Respiratory Distress Syndrome” in The New England Journal of Medicine 342 (2000) 1301-1308:
first algorithm: predicted weight [kg]=50+0.91*(height [cm]−130)
second algorithm: predicted weight [kg]=45.5+0.91 (height [cm]−130) and from Traub, Scott L. and Johnson, Cary E.: “Comparison of methods of estimating creatine clearance in children” in American Journal of Hospital Pharmacy 37 (1980) 195-201:
third algorithm: predicted weight [kg]=39+0.89·(height [cm]−152.4)
fourth algorithm: predicted weight [kg]=42.2+0.89·(height [cm]−152.4)
fifth algorithm: predicted weight [kg]=((height [cm])²·1.65)/1000
sixth algorithm: predicted weight [kg]=((height [cm])²·1.65)/1000

In this case, the first and second threshold values amount to 152.4 cm and are thus identical with each other and will be referred to simply as “adult threshold value” hereinafter. Likewise, the third and fourth threshold values amount to 152.4 cm and are thus identical with each other and will be referred to simply as “pediatric threshold value” hereinafter. It is to be understood, however, that varying threshold values may be used if they become available. Further, fifth and sixth algorithms are identical with each other and will be referred to simply as “fifth algorithm” hereinafter. As above, it is to be understood, however, that differing algorithms may be used if they are available.

It can also be seen that the above formulas on which the first, second, third and fourth algorithms are based, all share the form

predicted weight [kg]=A+B·(height [cm]−C)

Selecting the desired algorithm can thus be performed by simply assigning a certain value to the coefficient B or the constants A and C, respectively.

An algorithm selection procedure is schematically illustrated in FIG. 2.

Before determining normalised values of extravascular lung water EVLW, the operator is prompted, e.g. by a respective dialog box displayed on display 23, to input height and gender of the patient 3 and whether the patient 3 is an adult (adult mode) or a pediatric patient (pediatric mode)—step S1 in FIG. 2. For this input, a keyboard 1 or any other suitable input means (such as a touch screen, a track ball or the like) may be used.

The patient monitor 4 then allocates the patient 3 to a category corresponding to one of the above algorithms, as illustrated in FIG. 2, and determines the predicted weight (“predicted body weight” PBW) from the patient's 3 height using the respective algorithm (1st, 2nd, 3rd, 4th or 5th algorithm).

If it is determined (steps S2 and S3a) that adult mode is selected and the patient's 3 height is below the adult threshold value of—in the present example—130 cm (or the first or second threshold value, respectively), and thus outside the range for which the first and second algorithms are validated, the selection of adult mode is rejected (step S5) and it is left to a physician to decide whether the patient 3 is to be treated as a pediatric patient.

It is to be understood, however, that algorithms validated for adults with a height below the adult threshold value (or the first or second threshold value, respectively) may be used instead of rejecting the selection of the adult mode, if such algorithms are available.

If it is determined (steps S2 and S3a) that adult mode is selected and the patient's 3 height is equal to or above the adult threshold value of—in the present example—130 cm (or the first or second threshold value, respectively), and thus within the range for which the first and second algorithms are validated, the first algorithm is selected in case the gender is female and the second algorithm is selected in case the gender is male (step S4a)

If it is determined (steps S2 and S3b) that pediatric mode is selected and the patient's 3 height is equal to or above the pediatric threshold value of—in the present example—152.4 cm (or the third or fourth threshold value, respectively), and thus within the range for which the third and fourth algorithms are validated, the third algorithm is selected in case the gender is female and the fourth algorithm is selected in case the gender is male (step S4b)

If it is determined (steps S2 and S3b) that pediatric mode is selected and the patient's 3 height is below the pediatric threshold value of—in the present example—152.4 cm (or the third or fourth threshold value, respectively), and thus within the range for which the fifth algorithm is validated, the fifth algorithm is selected.

The predicted weight PBW is determined using the appropriate respective algorithm (step S51, S52, S53 S54, S55).

The extra vascular lung water determined as described above is then normalised by dividing by the predicted weight PBW (step S6). The normalised value EVLWi is displayed on the display 23 and/or otherwise output and may be stored in a memory or on a suitable external storage medium.

Further, global enddiastolic volume GEDV is normalised by dividing by a notional body surface area (“predicted body surface”) PBSA to yield the normalised value GEDVi.

GEDVi=GEDV/PBSA

The predicted body surface area PBSA can be determined, for example, using the following formula, if PBW is below 15 kg

PBSA=(PBW^{0.5378}·height [cm]^{0.3964})·0.24265

and using the following formula, if PBW is equal to or above 15 kg

PBSA=(PBW^{0.425}·height [cm]^{0.725})·0.007184

wherein PBW is the predicted body weight determined as described above.

Generally, a predicted body surface area can be determined by using a weight dependent known formula for body surface area and substituting actual weight with predicted body weight. The various algorithms for determining predicted body weight can thus be considered subalgorithms of respective algorithms for determining body surface area.

The thus normalised value is displayed on the display 23 and/or otherwise output and may be stored in a memory or on a suitable external storage medium.

1. A patient monitoring apparatus for determining a parameter representing an intrathoracic volume compartment of a patient (3), said patient monitoring apparatus comprising computing means adapted to
read in sensor data representing at least one physical quantity measured at a predetermined position of the patient's (3) cardiovascular system as a function of time, and to
determine said parameter representing said intrathoracic volume compartment using said sensor data,
said patient monitoring apparatus being characterised in that said patient monitoring apparatus further comprises input means (1) for inputting patient (3) specific information, said patient (3) specific information including categorizing information and biometric information,
and in that said computing means are further adapted
to allocate said patient (3) to a patient category depending on said categorizing information,
to select, depending on said patient category, an algorithm from a plurality of algorithms usable for normalising said parameter said intrathoracic volume compartment using said biometric information, and
to normalise said parameter representing said intrathoracic volume compartment using said algorithm.

2. A patient monitoring apparatus according to claim 1, wherein said intrathoracic volume compartment is extravascular lung water, intrathoracic blood volume, pulmonary blood volume or global enddiastolic volume.

3. A patient monitoring apparatus according to claim 1, wherein at least one of said physical quantities is a local blood temperature and said computing means are adapted to
process information characterizing an initial local temperature change in the proximity of another predetermined position of the patient's (3) cardiovascular system, and
to determine said parameter representing said intrathoracic volume compartment from said sensor data and said information characterizing said initial local temperature change using a thermodilution algorithm.

4. A patient monitoring apparatus according to claim 3, wherein said computing means are adapted to process information characterizing the initial local temperature change in such a manner that said computing means control effecting the initial local temperature change in accordance with said information.

5. A patient monitoring apparatus according to claim 3, wherein said computing means are adapted to read in the information characterizing the initial local temperature change for processing.

6. A patient monitoring apparatus according to claim 3, wherein said patient monitoring apparatus further comprises
temperature influencing means (11) for effecting the initial local temperature change in the proximity of said other position of the patient's (3) cardiovascular system, thus introducing a travelling temperature deviation to the patient's (3) blood stream, and
temperature sensing means (21) connected to said computing means for measuring said local blood temperature and providing said sensor data.

7. A patient monitoring apparatus according to claim 1, wherein said algorithms are algorithms for determining a notional weight of said patient (3) using said biometric information,
and said computing means are adapted to normalise said parameter representing said intrathoracic volume compartment with said notional weight.

8. A patient monitoring apparatus according to claim 1, wherein said algorithms are algorithms for determining a notional body surface area of said patient (3) using said biometric information,
and said computing means are adapted to normalise said parameter representing said intrathoracic volume compartment with said notional body surface area.

9. A patient monitoring apparatus according to claim 8, wherein said algorithms for determining a notional body surface area of said patient (3) comprise subalgorithms for determining a notional weight of said patient (3) using said biometric information.

10. A patient monitoring apparatus according to claim 1, wherein at least part of said biometric information is identical with at least part of said categorizing information.

11. A patient monitoring apparatus according to claim 1, wherein said biometric information includes said patient's (3) height.

12. A patient monitoring apparatus according to claim 1, wherein said categorizing information includes said patient's (3) gender.

13. A patient monitoring apparatus according to claim 1, wherein said categorizing information includes mode information, said mode information indicating whether said patient (3) is to be treated as an adult patient or as a pediatric patient, and said plurality of algorithms include at least one pediatric mode algorithm and at least one adult mode algorithm.

14. A patient monitoring apparatus according to claim 13, which is adapted to reject said mode information, if said mode information indicates that said patient (3) is to be treated as an adult patient and said biometric information includes a biometric value outside a predetermined range, for which said at least one adult mode algorithm is valid, or if said mode information indicates that said patient (3) is to be treated as a pediatric patient and said biometric information includes a biometric value outside a predetermined range, for which said at least one pediatric mode algorithm is valid.

15. A method of determining a parameter representing an intrathoracic volume compartment of a patient (3), wherein
sensor data is provided representing at least one physical quantity measured at a predetermined position of the patient's (3) cardiovascular system as a function of time, and
said parameter representing said intrathoracic volume compartment is determined using said sensor data,
said method being characterised in that it includes
acquiring patient specific information (S1), said patient specific information including categorizing information and biometric information,
allocating (S2, S3a, S3b, S4a, S4b) said patient (3) to a patient category depending on said categorizing information,
selecting, depending on said patient category, an algorithm from a plurality of algorithms usable for normalising said parameter representing said intrathoracic volume compartment using said biometric information, and
normalising (S51, S52, S53, S54, S55, S6) said parameter representing said intrathoracic volume compartment using said algorithm.

16. A method according to claim 15, wherein said intrathoracic volume compartment is extravascular lung water, intrathoracic blood volume, pulmonary blood volume or global enddiastolic volume.

17. A method according to claim 15, wherein at least one of said physical quantities is a local blood temperature and said parameter representing said intrathoracic volume compartment is determined from said sensor data and information characterizing an initial local temperature change in the proximity of another predetermined position of the patient's (3) cardiovascular system using a thermodilution algorithm.

18. A method according to claim 15, wherein said algorithms are algorithms for determining a notional weight of said patient (3) using said biometric information, and said parameter representing said intrathoracic volume compartment is normalised with said notional weight.

19. A method according to claim 15, wherein said algorithms are algorithms for determining a notional body surface area of said patient (3) using said biometric information,
and said parameter representing said intrathoracic volume compartment is normalised with said notional body surface area.

20. A method according to claim 15, wherein said algorithms for determining a notional body surface area of said patient (3) comprise subalgorithms for determining a notional weight of said patient (3) using said biometric information.

21. A method according to claim 15, wherein at least part of said biometric information is identical with at least part of said categorizing information.

22. A method according to claim 15, wherein said biometric information includes said patient's (3) height.

23. A method according to claim 15, wherein said categorizing information includes said patient's (3) gender.

24. A method according to claim 15, wherein said categorizing information includes mode information, said mode information indicating whether said patient (3) is to be treated as an adult patient or as a pediatric patient, and said plurality of algorithms include at least one pediatric mode algorithm and at least one adult mode algorithm.

25. A method according to claim 24, wherein said mode information is rejected (S5), if said mode information indicates that said patient (3) is to be treated as an adult patient and said biometric information includes a biometric value outside a predetermined range, for which said at least one adult mode algorithm is valid, or if said mode information indicates that said patient (3) is to be treated as a pediatric patient and said biometric information includes a biometric value outside a predetermined range, for which said at least one pediatric mode algorithm is valid.
